(19) United States
(12) Patent Application Publication
(10) Pub. No.: US 2005/0215611 A1
(43) Pub. Date: Sep. 29, 2005

(54) Combination therapy for treating cyclooxygenase-2 mediated diseases in patients at risk of thrombotic cardiovascular events

(76) Inventors: Richard H Hunt, Burlington (CA); Thomas J Simon, Bryn Mawr, PA (US)

Correspondence Address: MERCK AND CO., INC, P O BOX 2000, RAHWAY, NJ, 07065-0907, US

(21) Appl. No.: 10/513,085

(22) PCT Filed: May 9, 2003
(86) PCT No.: PCT/US03/14778

§ 371 Date: Oct. 28, 2004

Related U.S. Application Data
(60) Provisional application No. 60/388,660, filed on May 10, 2002.

(51) Int. Cl. A61K 31/415

(52) U.S. Cl. 514/406

(57) ABSTRACT

The present invention encompasses a method for treating a chronic cyclooxygenase-2 mediated disease or condition and reducing the risk of a thrombotic cardiovascular event in a human patient in need of such treatment and at risk of a thrombotic cardiovascular event comprising orally concomitantly or sequentially administering to said patient a cyclooxygenase-2 selective inhibitor in an amount effective to treat the cyclooxygenase-2 mediated disease or condition and nitric oxide releasing aspirin in an amount effective to reduce the risk of the thrombotic cardiovascular event while maintaining a high level of upper gastrointestinal safety and tolerability. The invention also encompasses a method for treating a chronic cyclooxygenase-2 mediated disease or condition and reducing the risk of a thrombotic cardiovascular event in a human patient in need of such treatment and at risk of a thrombotic cardiovascular event comprising orally concomitantly or sequentially administering to said patient a nitric oxide releasing-cyclooxygenase-2 selective inhibitor in an amount effective to treat the cyclooxygenase-2 mediated disease or condition and aspirin in an amount effective to reduce the risk of the thrombotic cardiovascular event while maintaining a high level of upper gastrointestinal safety and tolerability. Pharmaceutical compositions are also encompassed.

### CROSS REFERENCE TO RELATED APPLICATIONS

This application is a U.S. National Phase application under 35 U.S.C. § 371 of PCT Application No. PCT/US03/14778, filed May 9, 2003, which claims priority under 35 U.S.C. 119 to U.S. No. 60/388,660, filed May 10, 2002.

### BACKGROUND OF THE INVENTION

Selective inhibitors of cyclooxygenase-2 are a sub-class of the class of drugs known as non-steroidal antiinflammatory drugs (NSAIDs). The NSAIDs are active in reducing the prostaglandin-induced pain and swelling associated with the inflammation process but are also active in affecting other prostaglandin-regulated processes not associated with the inflammation process. Thus, use of high doses of most common NSAIDs can produce severe side effects, including life threatening ulcers, that limit their therapeutic potential. An alternative to NSAIDs is the use of corticosteroids, which have even more drastic side effects, especially when long term therapy is involved.

Previous NSAIDs have been found to prevent the production of prostaglandin by inhibiting enzymes in the human arachidonic acid/prostaglandin pathway including the enzyme cyclooxygenase (COX). The recent discovery that there are two isoforms of the COX enzyme, the first, COX-1, being involved with physiological functions and the second, COX-2, being induced in inflamed tissue, has given rise to a new approach. While conventional NSAIDs block both forms of the enzyme, the identification of the inducible COX-2 enzyme associated with inflammation has provided a viable target of inhibition which more effectively reduces inflammation and produces fewer and less drastic side effects. Many compounds which have activity as COX-2 inhibitors have been identified, including rofecoxib (VIOXX®), etoricoxib (ARCOXIA™), celecoxib (CELEBREX®) and valdecoxib (BEXTRA™), and much research continues in this area.

Many patients with a chronic cyclooxygenase-2 mediated disease or condition are elderly and thus are at increased risk for thrombotic cardiovascular events, such as stroke, myocardial ischemia, myocardial infarction, angina pectolis, transient ischemic attack (TIA; amaurosis fugax), reversible ischemic neurologic deficits, and any similar thrombotic event in any vascular bed (splanchnic, renal, aortic, peripheral, etc.). Moreover, there is evidence that patients with chronic inflammatory conditions, such as rheumatoid arthritis and systemic lupus erythematosis are at increased risk for thrombotic cardiovascular events. Thus, it is desirable that such patients receive appropriate antiplatelet therapy, such as aspirin, to reduce their risk of such events. However, very recent information from studies of the COX-2 selective inhibitors show that the incidence of gastric ulcer events in patients receiving both low-dose aspirin and the COX-2 selective inhibitor is similar to that seen with conventional NSAIDS that inhibit both COX-1 and COX-2. Also, some conventional NSAIDS, such as Naprosyn (naproxen sodium), when taken regularly, have been shown to have significant antiplatelet activity as well as efficacy in treating chronic cyclooxygenase-2 mediated diseases or conditions. Thus, the major advantage that COX-2 specific inhibitors have over NSAIDS (used in isolation or in combination with aspirin) may be substantially or completely offset by the concomitant use of aspirin.

NO-releasing aspirin is a modified version of aspirin that is reported to have improved antithrombtic effects as well as a reduced potential for gastrointestinal toxicity. See J. L. Wallace, et al., Nature reviews Drug Discovery, vol. 1, pp. 375-382, May 2002.

In the current invention, nitric oxide releasing aspirin is administered together with a cyclooxygenase-2 selective inhibitor or a nitric oxide releasing-cycloxygenase-2 selective inhibitor is administered together with aspirin leading to several advantages. First, the combination reduces the risk of GI ulcers and bleeds (which is otherwise particularly increased when both a COX-2 inhibitor and aspirin are administered on a daily basis) while providing substantial antiplatelet efficacy. Second, the concomitant therapy provides symptom relief for the chronic cyclooxygenase-2 mediated disease or condition, such as chronic pain or arthritis.

Thus, the invention provides for a clearly superior profile than that hitherto obtainable in that it provides efficacy in treating chronic cyclooxygenase-2 mediated diseases or conditions, effectively inhibits platelets thus reducing the risk of thrombotic cardiovascular events and at the same time reduces the risk of GI ulceration or bleeding relative either to conventional NSAIDS or separate administration of a COX-2 inhibitor and low-dose daily aspirin, or than a NSAID plus aspirin.

### SUMMARY OF THE INVENTION

The present invention encompasses a method for treating a chronic cyclooxygenase-2 mediated disease or condition and reducing the risk of a thrombotic cardiovascular event in a human patient in need of such treatment and at risk of a thrombotic cardiovascular event comprising orally concomitantly or sequentially administering to said patient a cyclooxygenase-2 selective inhibitor in an amount effective to treat the cyclooxygenase-2 mediated disease or condition and nitric oxide releasing aspirin in an amount effective to reduce the risk of the thrombotic cardiovascular event while maintaining a high level of upper gastrointestinal safety and tolerability. The invention also encompasses a method for treating a chronic cyclooxygenase-2 mediated disease or condition and reducing the risk of a thrombotic cardiovascular event in a human patient in need of such treatment and at risk of a thrombotic cardiovascular event comprising orally concomitantly or sequentially administering to said patient a nitric oxide releasing-cyclooxygenase-2 selective inhibitor in an amount effective to treat the cyclooxygenase-2 mediated disease or condition and aspirin in an amount effective to reduce the risk of the thrombotic cardiovascular event while maintaining a high level of upper gastrointestinal safety and tolerability. Pharmaceutical compositions are also encompassed.

### DETAILED DESCRIPTION OF THE INVENTION

The present invention encompasses a method for treating a chronic cyclooxygenase-2 mediated disease or condition and reducing the risk of a thrombotic cardiovascular event in a human patient in need of such treatment and at risk of a thrombotic cardiovascular event comprising orally concomitantly or sequentially administering to said patient a cyclooxygenase-2 selective inhibitor in an amount effective to treat the cyclooxygenase-2 mediated disease or condition and nitric oxide releasing aspirin in an amount effective to reduce the risk of the thrombotic cardiovascular event while maintaining a high level of upper gastrointestinal safety and tolerability. The invention also encompasses a method for treating a chronic cyclooxygenase-2 mediated disease or condition and reducing the risk of a thrombotic cardiovascular event in a human patient in need of such treatment and at risk of a thrombotic cardiovascular event comprising orally concomitantly or sequentially administering to said patient a nitric oxide releasing-cyclooxygenase-2 selective inhibitor in an amount effective to treat the cyclooxygenase-2 mediated disease or condition and aspirin in an amount effective to reduce the risk of the thrombotic cardiovascular event while maintaining a high level of upper gastrointestinal safety and tolerability.

The term “treating a chronic cylcooxygenase-2 mediated disease or condition” means treating or preventing any chronic disease or condition that is advantageously treated or prevented by inhibiting the cyclooxygenase-2 enzyme. The term includes the relief of pain, fever and inflammation of a variety of conditions including rheumatic fever, symptoms associated with influenza or other viral infections, common cold, low back pain, neck pain, dysmenorrhea, headache, migraine, toothache, sprains and strains, myositis, neuralgia, synovitis, arthritis, including rheumatoid arthritis, degenerative joint diseases (osteoarthritis), gout, ankylosing spondylitis, bursitis, burns, injuries, and pain and inflammation following surgical procedures. In addition, such a compound may inhibit cellular neoplastic transformations and metastatic tumor growth and hence can be used in the treatment and/or prevention of cancer. In addition, such a compound may inhibit the onset or progression of Altzheimer's disease or cognitive impairment. The term also includes the treatment and/or prevention of cyclooxygenase-mediated proliferative disorders such as may occur in diabetic retinopathy and tumor angiogenesis. The term “treating” encompasses not only treating a patient to relieve the patient of the signs and symptoms of the disease or condition but also prophylactically treating an asymptomatic patient to prevent the onset or progression of the disease or condition.

A “thrombotic cardiovascular event” is defined as any sudden event of a type known to be caused by platelet aggregation, thrombosis, and subsequent ischemic clinical events, including thrombotic or thromboembolic stroke, myocardial ischemia, myocardial infarction, angina pectoris, transient ischemic attack (TIA; amaurosis fugax), reversible ischemic neurologic deficits, and any similar thrombotic event in any vascular bed (splanchnic, renal, aortic, peripheral, etc.).

The term “patient in need of such treatment and at risk of a thrombotic cardiovascular event” means a patient in need of both treatment for a cyclooxygenase-2 mediated disease and also at risk of a thrombotic cardiovascular event. One skilled in the art can diagnose a patient that is in need of treatment for a cyclooxygenase-2 mediated disease or condition and also at risk of suffering a thrombotic cardiovascular event. For example, such a patient may be over the age of 50 with osteoarthritis and with a previous myocardial infarction. Other risk factors for a thrombotic cardiovascular event include hypertension, hypercholesterolemia, diabetes mellitus, chronic renal impairment, smoking, and any prior personal or family history of such an event. Administration of the drug combination to the patient includes both self-administration and administration to the patient by another person.

The terms “nitric oxide releasing-aspirin” and “NO-aspirin” mean a modified version of aspirin linked to a NO releasing moiety by means of a linking group such as an ester linkage. Such compounds are known in the art and disclosed, for example, in J. L. Wallace, et al., Nature Reviews Drug Discovery, vol. 1, pp. 375-382, May 2002, which is hereby incorporated by reference in its entirety. See the compound referred therein as NCX4016. Other examples of nitric oxide releasing NSAIDs are well known in the art and are disclosed, for example, in the following patents and published applications which are hereby incorporated by reference in their entirety: U.S. Pat. No. 6,323,234, issued on Nov. 27, 2001; U.S. Pat. No. 6,297,260, issued on Oct. 2, 2001; U.S. Pat. No. 6,143,734, issued on Nov. 7, 2000; U.S. Pat. No. 6,083,515, issued on Jul. 4, 2000; U.S. Pat. No. 6,057,347, issued on May 2, 2000; U.S. Pat. No. 6,048,858, issued on Apr. 11, 2000; U.S. Pat. No. 6,043,233, issued on Mar. 28, 2000; U.S. Pat. No. 6,043,232, issued Mar. 28, 2000; U.S. Pat. No. 5,780,495, issued Jul. 14, 1998; U.S. Pat. No. 5,703,073, issued Dec. 30, 1997; U.S. Pat. No. 5,700,947, issued Dec. 23, 1997; U.S. Pat. No. 5,621,000, issued Apr. 15, 1997; WO 00/25776, published on May 11, 2000; WO 96/32946, published on Oct. 24, 1996; and WO 95/09831, published on Apr. 13, 1995. See also, Bandarage, et al., J. Med. Chem., vol. 43, pp. 4005-4016, 2000; Wallace, et al., Drug Development Research, vol. 42, pp. 144-149, 1997; Wallace, et al., Gastroenterology, vol. 107, pp. 173-179, 1994; and Wallace, et al., European Journal of Pharmacology, vol. 257, pp. 249-255, 1994, all of which are hereby incorporated by reference in their entirety.

The terms “inhibitor of cyclooxygenase-2”, “cyclooxygenase-2 selective inhibitor” and “COX-2 inhibitor” as used herein embrace compounds which selectively inhibit cyclooxygenase-2 over cyclooxygenase-1. Employing the human whole blood COX-1 assay and the human whole blood COX-2 assay described in C. Brideau et al, Inflamm. Res. 45: 68-74 (1996), herein incorporated by reference, preferably, the compounds have a cyclooxygenase-2 IC₅₀ of less than about 2 μM in the human whole blood COX-2 assay, yet have a cyclooxygenase-1 IC₅₀ of greater than about 5 μM in the human whole blood COX-1 assay. Also preferably, the compounds have a selectivity ratio of cyclooxygenase-2 inhibition over cyclooxygenase-1 inhibition of at least 10, and more preferably of at least 40. The resulting selectivity may indicate an ability to reduce the incidence of common NSAID-induced side effects, especially erosions and ulceration of the upper gastrointestinal mucosa.

Examples of cyclooxygenase-2 selective inhibitors include rofecoxib (VIOXX®, see U.S. Pat. No. 5,474,995, hereby incorporated by reference in its entirety), etoricoxib (ARCOXA™ see U.S. Pat. No. 5,861,419, hereby incorporated by reference in its entirety), celecoxib (CELEBREX®, see U.S. Pat. No. 5,466,823, hereby incorporated by reference in its entirety), valdecoxib (see U.S. Pat. No. 6,633,272, hereby incorporated by reference in its entirety), parecoxib (see U.S. Pat. No. 5,932,598, hereby incorporated by reference in its entirety), COX-189 (Novartis), BMS347070 (Bristol Myers Squibb), tiracoxib or JTE522 (Japan Tobacco), ABT963 (Abbott), CS502 (Sankyo) and GW406381 (GlaxoSmithKline).

The terms “nitric oxide releasing-cyclooxygenase-2 selective inhibitor,” “NO-cyclooxygenase-2 selective inhibitor,” “nitric oxide releasing-COX-2 inhibitor” and “NO-COX-2 inhibitor” mean a modified version of a cycloxygenase-2 selective inhibitor as defined above linked to a NO releasing moiety by means of a linking group such as an ester linkage. Examples of such compounds are known in the art and disclosed, for example, in WO01/45703, published on Jun. 28, 2001, which is hereby incorporated by reference in its entirety.

The term “amounts that are effective to treat” is intended to mean that amount of a drug or pharmaceutical agent that will elicit the biological or medical response of a tissue, a system, animal or human that is being sought by a researcher, veterinarian, medical doctor or other clinician. The term also encompasses the amount of a pharmaceutical drug that will prevent or reduce the risk of occurrence of the biological or medical event that is sought to be prevented in a tissue, a system, animal or human by a researcher, veterinarian, medical doctor or other clinician. The inhibitor of cyclooxygenase-2 may be administered at a dosage level up to conventional dosage levels for NSAIDs. Suitable dosage levels will depend upon the antiinflammatory effect of the chosen inhibitor of cyclooxygenase-2, but typically suitable levels will be about 0.001 to 50 mg/kg per day, preferably 0.005 to 30 mg/kg per day, and especially 0.05 to 10 mg/kg per day. The compound may be administered on a regimen of once or twice per day.

The term “amount effective to reduce the risk of means the amount of a pharmaceutical drug that will prevent or reduce the risk of occurrence of the biological or medical event that is sought to be prevented in a tissue, a system, animal or human by a researcher, veterinarian, medical doctor or other clinician. Aspirin is administered at a dose of about 30 mg to about 1 g once daily, preferably at a dose of about 80 mg to about 650 mg.

The term “concomitantly administering” means administering the agents substantially concurrently. The term “concomitantly administering” encompasses not only administering the two agents in a single pharmaceutical dosage form but also the administration of each active agent in its own separate pharmaceutical dosage formulation. Where separate dosage formulations are used, the agents can be administered at essentially the same time, i.e., concurrently.

The term “sequentially administering” means administering the agents at separately staggered times. Thus, agents can be sequentially administered such that the beneficial pharmaceutical effect of NO-aspirin and the COX-2 inhibitor or aspirin and the NO-COX-2 inhibitor are realized by the patient at substantially the same time. Thus, for example, if a COX-2 selective inhibitor and NO releasing aspirin are both administered on a once a day basis, the interval of separation between sequential administration of the two agents can be up to twelve hours apart.

The invention encompasses a method for treating a chronic cyclooxygenase-2 mediated disease or condition and reducing the risk of a thrombotic cardiovascular event in a human patient in need of such treatment and at risk of a thrombotic cardiovascular event comprising orally concomitantly or sequentially administering to said patient a cyclooxygenase-2 selective inhibitor in an amount effective to treat the cyclooxygenase-2 mediated disease or condition and nitric oxide releasing aspirin in an amount effective to reduce the risk of the thrombotic cardiovascular event. Within this embodiment is encompassed the above method wherein the cyclooxygenase-2 selective inhibitor is selected from the group consisting of: rofecoxib, etoricoxib, celecoxib, valdecoxib, COX-189, BMS347070, tiracoxib, ABT963, CS502 and GW406381. Also, within this embodiment is encompassed the above method wherein the cyclooxygenase-2 selective inhibitor is rofecoxib. Also, within this embodiment is encompassed the above method wherein rofecoxib is administered on a once or twice daily basis at a dose of about 12.5 mg or about 25 mg. Also, within this embodiment is encompassed the above method wherein rofecoxib is administered on a once daily basis. Also, within this embodiment is encompassed the above method wherein the cyclooxygenase-2 selective inhibitor is etoricoxib. Also, within this embodiment is encompassed the above method wherein etoricoxib is administered on a once or twice daily basis at a dose of about 60 mg, about 90 mg or about 120 mg. Also, within this embodiment is encompassed the above method wherein the cyclooxygenase-2 selective inhibitor is celecoxib. Also, within this embodiment is encompassed the above method wherein wherein celecoxib is administered on a once or twice daily basis at a dose of about 100 mg or about 200 mg or 400 mg. Also, within this embodiment is encompassed the above method wherein the cyclooxygenase-2 selective inhibitor is valdecoxib. Also, within this embodiment is encompassed the above method wherein valdecoxib is administered on a once or twice daily basis at a dose of about 10 mg or about 20 mg. Also, within this embodiment is encompassed the above method wherein wherein the cyclooxygenase-2 selective inhibitor is administered orally on a once daily basis. Also, within this embodiment is encompassed the above method wherein wherein the cyclooxygenase-2 selective inhibitor is administered orally on a twice daily basis.

Another embodiment of the invention encompasses the above method wherein the cyclooxygenase-2 selective mediated disease or condition is osteoarthritis. Another embodiment of the invention encompasses the above method wherein the cyclooxygenase-2 selective mediated disease or condition is rheumatoid arthritis. Another embodiment of the invention encompasses the above method wherein the cyclooxygenase-2 selective mediated disease or condition is chronic pain.

The invention also encompasses the above method wherein nitric oxide releasing aspirin is administered at a dose of about 30 mg to about 1 g. Another embodiment of the invention encompasses the above method wherein nitric oxide releasing aspirin is administered at a dose of about 80 to about 650 mg. Another embodiment of the invention encompasses the above method wherein nitric oxide releasing aspirin is administered at a dose of about 81 mg. Another embodiment of the invention encompasses the above method wherein nitric oxide releasing aspirin is administered at a dose of about 325 mg. Another embodiment of the invention encompasses the above method wherein aspirin is orally administered once daily.

The invention also encompasses a method for treating a chronic cyclooxygenase-2 mediated disease or condition and reducing the risk of a thrombotic cardiovascular event in a human patient in need of such treatment and at risk of a thrombotic cardiovascular event comprising orally concomitantly or sequentially administering to said patient a nitric oxide releasing-cyclooxygenase-2 selective inhibitor in an amount effective to treat the cyclooxygenase-2 mediated disease or condition and aspirin in an amount effective to reduce the risk of the thrombotic cardiovascular event. Also within this embodiment is encompassed the above method wherein the nitric oxide releasing-cyclooxygenase-2 selective inhibitor is administered orally on a once daily basis. Also within this embodiment is encompassed the above method wherein the nitric oxide releasing-cyclooxygenase-2 selective inhibitor is administered orally on a twice daily basis.

Another embodiment of the invention encompasses the above method wherein the cyclooxygenase-2 selective mediated disease or condition is osteoarthritis. Another embodiment of the invention encompasses the above method wherein the cyclooxygenase-2 selective mediated disease or condition is rheumatoid arthritis. Another embodiment of the invention encompasses the above method wherein the cyclooxygenase-2 selective mediated disease or condition is chronic pain.

The invention also encompasses the above method wherein aspirin is administered at a dose of about 30 mg to about 1 g. Another embodiment of the invention encompasses the above method wherein aspirin is administered at a dose of about 80 to about 650 mg. Another embodiment of the invention encompasses the above method wherein aspirin is administered at a dose of about 81 mg. Another embodiment of the invention encompasses the above method wherein aspirin is administered at a dose of about 325 mg. Another embodiment of the invention encompasses the above method wherein aspirin is orally administered once daily.

The invention also encompasses a pharmaceutical composition comprising a cyclooxygenase-2 selective inhibitor and nitric oxide releasing aspirin in combination with a pharmaceutically acceptable carrier. Within this embodiment is encompassed the pharmaceutical composition wherein the nitric oxide releasing aspirin is present in an amount ranging from about 80 to about 650 mg. Also within this embodiment is encompassed the pharmaceutical composition wherein the cyclooxygenase-2 selective inhibitor is rofecoxib. Also within this embodiment is encompassed the pharmaceutical composition wherein rofecoxib is present in an amount of about 12.5 mg or about 25 mg. Also within this embodiment is encompassed the pharmaceutical composition wherein the cyclooxygenase-2 selective inhibitor is etoricoxib. Also within this embodiment is encompassed the pharmaceutical composition wherein etoricoxib is present at a dose of about 60 mg, about 90 mg or about 120 mg.

The invention also encompasses a pharmaceutical composition comprising a nitric oxide releasing-cyclooxygenase-2 selective inhibitor and aspirin in combination with a pharmaceutically acceptable carrier. Within this embodiment is encompassed the pharmaceutical composition wherein aspirin is present in an amount ranging from about 80 to about 650 mg.

The present invention also encompasses a method for treating a chronic cyclooxygenase-2 mediated disease or condition and reducing the combined risk of death and nonfatal stroke in a human patient who has had ischemic stroke of transient ischemia of the brain due to fibrin platelet emboli and also in need of treatment for a chronic cyclooxygenase-2 mediated disease comprising:
1) orally concomitantly or sequentially administering to said patient a cyclooxygenase-2 selective inhibitor in an amount effective to treat the cyclooxygenase-2 mediated disease or condition and nitric oxide releasing aspirin in an amount effective to reduce the combined risk of death and nonfatal stroke, or
2) orally concomitantly or sequentially administering to said patient a nitric oxide releasing-cyclooxygenase-2 selective inhibitor in an amount effective to treat the cyclooxygenase-2 mediated disease or condition and aspirin in an amount effective to reduce the combined risk of death and nonfatal stroke in a human patient who has had ischemic stroke of transient ischemia of the brain due to fibrin platelet emboli.

The present invention also encompasses a method for treating a chronic cyclooxygenase-2 mediated disease or condition and reducing the combined risk of death and nonfatal myocardial infarction in a human patient with a previous myocardial infarction or unstable angina pectoris and also in need of treatment for a chronic cyclooxygenase-2 mediated disease comprising:
1) orally concomitantly or sequentially administering to said patient a cyclooxygenase-2 selective inhibitor in an amount effective to treat the cyclooxygenase-2 mediated disease or condition and nitric oxide releasing aspirin in an amount effective to reduce the combined risk of death and nonfatal myocardial infarction, or
2) orally concomitantly or sequentially administering to said patient a nitric oxide releasing-cyclooxygenase-2 selective inhibitor in an amount effective to treat the cyclooxygenase-2 mediated disease or condition and aspirin in an amount effective to reduce the combined risk of death and nonfatal myocardial infarction.

The present invention also encompasses a method for treating a chronic cyclooxygenase-2 mediated disease or condition and reducing the combined risk of death and nonfatal myocardial infarction in a human patient with chronic stable angina pectoris and also in need of treatment for a chronic cyclooxygenase-2 mediated disease comprising
1) orally concomitantly or sequentially administering to said patient a cyclooxygenase-2 selective inhibitor in an amount effective to treat the cyclooxygenase-2 mediated disease or condition and nitric oxide releasing aspirin in an amount effective to reduce the combined risk of death and nonfatal myocardial infarction, or
2) orally concomitantly or sequentially administering to said patient a nitric oxide releasing-cyclooxygenase-2 selective inhibitor in an amount effective to treat the cyclooxygenase-2 mediated disease or condition and aspirin in an amount effective to reduce the combined risk of death and nonfatal myocardial infarction.

For purposes of this specification, references to the compounds of use in this invention are meant to also include the pharmaceutically acceptable salts.

The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosarnine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

The compounds of use in this invention may have one or more chiral centers and the present compounds may occur as racemates, racemic mixtures and as individual diasteriomers or enantiomers with all such isomeric forms and mixtures thereof being included within the scope of this invention. Furthermore, some of the crystalline forms for compounds of the present invention may exist as polymorphs and as such are intended to be included in the present invention. In addition, some of the compounds of the instant invention may form solvates with water or common organic solvents. Such solvates and hydrates, as well as anhydrous compositions, are encompassed within the scope of this invention. Some of the compounds described herein may contain olefinic double bonds, and unless specified otherwise, are meant to include both E and Z geometric isomers.

The COX-2 inhibitors or NO-COX-2 inhibitors that may be used with this invention encompass all pharmaceutically acceptable salt forms of the compounds. Examples of such salt forms include but are not limited to salts derived from inorganic bases including aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosarnine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When treating a patient following the methods of the present invention, the combination may be administered on the same day. Thus, the instant pharmaceutical combination includes administration of a single pharmaceutical dosage formulation which contains both NO-aspirin and the COX-2 inhibitor or aspirin and the NO-COX-2 inhibitor, as well as administration of each active agent in its own separate pharmaceutical dosage formulation. Where separate dosage formulations are used, the agents can be administered at essentially the same time, i.e., concurrently, or at separately staggered times, i.e., sequentially. The instant pharmaceutical combination is understood to include all these regimens. Administration in these various ways are suitable for the present invention as long as the beneficial pharmaceutical effect of NO-aspirin and the COX-2 inhibitor or aspirin and the NO-COX-2 inhibitor are realized by the patient at substantially the same time. The dosage regimen utilizing the instant combination therapy is selected in accordance with a variety of factors including type, species, age, weight, sex and medical condition of the patient; the severity of the condition to be treated; the route of administration; the renal and hepatic function of the patient; and the particular compound or salt or ester thereof employed.

In the methods of the present invention, the active agents are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants (BHA, BHT, propyl gallate, sodium ascorbate, citric acid) can also be added to stabilize the dosage forms. Other suitable components include gelatin, sweeteners, natural and synthetic gums such as acacia, tragacanth or alginates, carboxymethylcellulose, polyethylene glycol, waxes and the like.

The active drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Active drug may also be delivered by the use of monoclonal antibodies as individual carriers to which the compound molecules are coupled. Active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The instant invention also encompasses a process for preparing a pharmaceutical composition comprising combining nitric oxide releasing-aspirin and the COX-2 inhibitor with a pharmaceutically acceptable carrier, as well as the pharmaceutical composition which is made by combining nitric oxide releasing-aspirin and the COX-2 inhibitor with a pharmaceutically acceptable carrier. The instant invention also encompasses a process for preparing a pharmaceutical composition comprising combining aspirin and the nitric oxide releasing-COX-2 inhibitor with a pharmaceutically acceptable carrier, as well as the pharmaceutical composition which is made by combining aspirin and the nitric oxide releasing-COX-2 inhibitor with a pharmaceutically acceptable carrier.

A therapeutically effective amount of nitric oxide releasing-aspirin and a COX-2 inhibitor can be used together for the preparation of a medicament useful for treating or preventing the disease or conditions herein. For example, the medicament may be comprised of a COX-2 inhibitor in combination with about 30 mg to 1 g of nitric oxide releasing-aspirin, or more particularly about 80 mg to about 650 mg of nitric oxide releasing-aspirin. A therapeutically effective amount of aspirin and a nitric oxide releasing-COX-2 inhibitor can be used together for the preparation of a medicament useful for treating or preventing the disease or conditions herein. For example, the medicament may be comprised of a nitric oxide releasing-COX-2 inhibitor in combination with about 30 mg to 1 g of aspirin, or more particularly about 80 mg to about 650 mg of aspirin.

The instant invention also encompasses the use of nitric oxide releasing-aspirin for the preparation of a medicament for the combined use with a cyclooxygenase-2 inhibitor for use as provided by the present invention; and the use of a cyclooxygenase-2 inhibitor for the preparation of a medicament for the combined use with nitric oxide releasing-aspirin for use as provided by the present invention.

The instant invention also encompasses the use of aspirin for the preparation of a medicament for the combined use with a nitric oxide releasing-cyclooxygenase-2 inhibitor for use as provided by the present invention; and the use of a nitric oxide releasing-cyclooxygenase-2 inhibitor for the preparation of a medicament for the combined use with aspirin for use as provided by the present invention.

1-21. (canceled)

22. A method for treating a chronic cyclooxygenase-2 mediated disease or condition and reducing the risk of a thrombotic cardiovascular event in a human patient in need of such treatment and at risk of a thrombotic cardiovascular event comprising orally concomitantly or sequentially administering to said patient a nitric oxide releasing-cyclooxygenase-2 selective inhibitor in an amount effective to treat the cyclooxygenase-2 mediated disease or condition and aspirin in an amount effective to reduce the risk of the thrombotic cardiovascular event.

23. The method according to claim 22 wherein the nitric oxide releasing-cyclooxygenase-2 selective inhibitor is administered orally on a once daily basis.

24. The method according to claim 22 wherein the nitric oxide releasing-cyclooxygenase-2 selective inhibitor is administered orally on a twice daily basis.

25. The method according to claim 22 wherein the cyclooxygenase-2 selective mediated disease or condition is osteoarthritis.

26. The method according to claim 22 wherein the cyclooxygenase-2 selective mediated disease or condition is rheumatoid arthritis.

27. The method according to claim 22 wherein the cyclooxygenase-2 selective mediated disease or condition is chronic pain.

28. The method according to claim 22 wherein aspirin is administered at a dose of about 30 mg to about 1 g.

29. The method according to claim 28 wherein aspirin is administered at a dose of about 80 to about 650 mg.

30. The method according to claim 29 wherein aspirin is administered at a dose of about 81 mg.

31. The method according to claim 29 wherein aspirin is administered at a dose of about 325 mg.

32. The method according to claim 22 wherein aspirin is orally administered once daily.

33-38. (canceled)

39. A pharmaceutical composition comprising a nitric oxide releasing-cyclooxygenase-2 selective inhibitor and aspirin in combination with a pharmaceutically acceptable carrier.

40. The pharmaceutical composition according to claim 39 wherein aspirin is present in an amount ranging from about 80 to about 650 mg.
